(19) United States
(12) Patent Application Publication
(10) Pub. No.: US 2024/0066158 A1
(43) Pub. Date: Feb. 29, 2024

(54) SYSTEM AND METHOD FOR IRRADIATING CANNABIS TO MITIGATE BIOLOGICAL CONTAMINATION

(71) Applicant: VJ ELECTRONIX, Bohemia, NY (US)

(72) Inventors: Donald Howard Naugler, Westford, MA (US); Mark E. Clemons, Weston, MA (US); Anton Krassilnikov, Littleton, MA (US)

(21) Appl. No.: 17/894,406

(22) Filed: Aug. 24, 2022

(51) Int. Cl. A61L 2/00; A61L 2/24; A61L 2/26

(52) U.S. Cl. CPC A61L 2/0041; A61L 2/24; A61L 2/26; A61L 2202/11; A61L 2202/14; A61L 2202/21

(57) ABSTRACT

An X-ray irradiation system includes a support for an object requiring decontamination, a first source of irradiation directed to a top side of the object; a second source of irradiation directed to a bottom side of the object; and a controller configured to control a dose rate and a time of each of the first source and the second source to optimize decontamination of the object. A method of irradiating an object is further disclosed.

### BACKGROUND

### 1. Field of the Disclosure

At least one example in accordance with the present disclosure relates generally to X-ray irradiation systems.

### 2. Discussion of Related Art

The X-ray is generally considered to be defined by wavelengths ranging from about 10⁻⁸ to 10⁻¹² meters and corresponding frequencies from about 10¹⁶ to 10²⁰ hertz. Machines harnessing the power of the X-ray to image the human body and detect objects outside the wavelengths visible to the human eye began soon after the X-ray itself was discovered in the late nineteenth century by German scientist Wilhelm Conrad Röntgen. Since that time, X-ray technology has been used for a variety of other purposes, such as treating products for bacteria and mold. For example, X-ray technology has been found to be effective in decontaminating biological or organic materials, such as food products, pharmaceuticals, and cannabis. For example, cannabis products can suffer from mold and bacteria contamination, which requires eradication prior to being sold. Irradiation from X-ray exposure has been found to eliminate such contamination.

### SUMMARY

One aspect of the present disclosure is directed to an X-ray irradiation system comprising a support for an object requiring decontamination, a first source of irradiation directed to a top side of the object; a second source of irradiation directed to a bottom side of the object; and a controller configured to control a dose rate and a time of each of the first source and the second source to optimize decontamination of the object.

Embodiments of the X-ray irradiation system further may include configuring the first source of irradiation to produce a dose rate of approximately 3.4 Gy/min at the point of entry (top) of the object and a dose rate of approximately 0.7 Gy/min at a depth of 18-inches (bottom) of the object and configuring the second source of irradiation to produce a dose rate of approximately 3.4 Gy/min at the point of entry (bottom) of the object and a dose rate is approximately 2.95 Gy/min at a depth of 0-inches (top) of the object. A combined dose rate of the first source of irradiation and the second source of irradiation may result in a dose rate between approximately 2.95 Gy/min to approximately 4.0 Gy/min through the object. The object may be a cannabis mass. The first source of irradiation may be configured to direct irradiation to a top surface of the cannabis mass and the second source of irradiation may be configured to direct irradiation to a bottom surface of the cannabis mass. The first source of irradiation and the second source of irradiation may each be configured to be adjusted to adjust an angle of the source of irradiation with respect to the object. The first source of irradiation and the second source of irradiation may be oriented in opposite directions. A combined dose rate of the first source of irradiation and the second source of irradiation may result in a dose rate that is more uniform and provides nearly double the dose rate of the first source of irradiation or the second source of irradiation. The X-ray irradiation system further may include a dose probe configured to monitor the object to obtain a delivered total dose applied to the object. At least one of the first source of irradiation and the second source of irradiation may be configured to be adjusted to adjust a distance of the source of irradiation with respect to the object. Both the first source of irradiation and the second source of irradiation may be configured to be adjusted to adjust the distance of the source of irradiation with respect to the object. A process speed may be optimized when adjusting the distance of at least one of the first source of irradiation and the second source of irradiation.

Another aspect of the present disclosure is directed to a method of irradiating an object with an X-ray irradiation system. In one embodiment, the method comprises: applying a first source of irradiation directed to a top side of the object; applying a second source of irradiation directed to a bottom side of the object; and controlling a dose rate and a time of each of the first source and the second source to optimize decontamination of the object.

Embodiments of the method further may include when applying the first source of irradiation, applying a dose rate of approximately 3.4 Gy/min at the point of entry (top) of the object and a dose rate of approximately 0.7 Gy/min at a depth of 18-inches (bottom) of the object and applying the second source of irradiation includes a dose rate of approximately 3.4 Gy/min at the point of entry (bottom) of the object and a dose rate is approximately 0.7 Gy/min at a depth of 0-inches (top) of the object. A combined dose rate of the first source of irradiation and the second source of irradiation may result in a dose rate between approximately 2.95 Gy/min to approximately 4.0 Gy/min through the object. The object may be a cannabis mass. Applying the first source of irradiation may include directing irradiation to a top surface of the cannabis mass and applying the second source of irradiation may include directing irradiation to a bottom surface of the cannabis mass. The method further may include adjusting an angle of one of the first source of irradiation and the second source of irradiation with respect to the object. The first source of irradiation and the second source of irradiation may be oriented in opposite directions. A combined dose rate of the first source of irradiation and the second source of irradiation may result in a dose rate that is more uniform and provides nearly double the dose rate of the first source of irradiation or the second source of irradiation. The method further may include positioning a dose in the object to monitor the object to obtain a delivered total dose applied to the object. The method further may include adjusting a distance of at least one of the first source of irradiation and the second source of irradiation with respect to the object. The further may include optimizing a process speed when adjusting the distance of at least one of the first source of irradiation and the second source of irradiation.

Still other aspects, embodiments, and advantages of these exemplary aspects and embodiments are discussed in detail below. Embodiments disclosed herein may be combined with other embodiments in any manner consistent with at least one of the principles disclosed herein, and references to “an embodiment,” “some embodiments,” “an alternate embodiment,” “various embodiments,” “one embodiment” or the like are not necessarily mutually exclusive and are intended to indicate that a particular feature, structure, or characteristic described may be included in at least one embodiment. The appearances of such terms herein are not necessarily all referring to the same embodiment.

### BRIEF DESCRIPTION OF THE DRAWINGS

Various aspects of at least one embodiment are discussed below with reference to the accompanying figures, which are not intended to be drawn to scale. The figures are included to provide an illustration and a further understanding of the various aspects and embodiments, and are incorporated in and constitute a part of this specification, but are not intended as a definition of the limits of any particular embodiment. The drawings, together with the remainder of the specification, serve to explain principles and operations of the described and claimed aspects and embodiments. In the figures, each identical or nearly identical component that is illustrated in various figures is represented by a like numeral. For purposes of clarity, not every component may be labeled in every figure. In the figures:

FIG. 1 is a side elevational view with packaging removed of an exemplary X-ray imaging system;

FIG. 2 is a schematic view of an X-ray irradiation system of an embodiment of the present disclosure configured to perform two-sided irradiation on a product;

FIG. 3 is a graph showing dose rate in cannabis from a single source on a single side of a package of cannabis;

FIG. 4 is a schematic representation of X-ray irradiation applied to the single side of the package of cannabis;

FIG. 5 is a graph showing dose rate in cannabis from two sources applied on two sides of a package of cannabis;

FIG. 6 is a schematic representation of X-ray irradiation applied to two sides of the package of cannabis;

FIG. 7 is a schematic representation of X-ray irradiation energy intensity levels as an incident of beam orientation;

FIG. 8 is a graph showing dose versus beam angle;

FIG. 9 is a graph showing total dose versus beam angle;

FIG. 10 is a graph showing full depth dose rate from two sources for various depths of product;

FIG. 11 is a graph showing time to target dose for various depth of product;

FIG. 12 is a graph showing three-inch depth dose rate;

FIG. 13 is a schematic representation of X-ray irradiation applied to two sides of a package of cannabis;

FIG. 14 is a graph showing dose rate for product in a twelve-inch tray or bin; and

FIG. 15 is a schematic representation of X-ray irradiation applied to two sides of a package of cannabis.

### DETAILED DESCRIPTION

Examples of the supports and X-ray irradiation systems described herein are not limited in application to the details of construction and the arrangement of components set forth in the following description or illustrated in the accompanying drawings. The X-ray irradiation systems are capable of implementation in other embodiments and of being practiced or of being carried out in various ways. Examples of specific implementations are provided herein for illustrative purposes only and are not intended to be limiting. In particular, acts, components, elements and features discussed in connection with any one or more examples are not intended to be excluded from a similar role in any other examples.

Also, the phraseology and terminology used herein is for the purpose of description and should not be regarded as limiting. Any references to examples, embodiments, components, elements or acts of the systems and methods herein referred to in the singular may also embrace embodiments including a plurality, and any references in plural to any embodiment, component, element or act herein may also embrace embodiments including only a singularity. References in the singular or plural form are not intended to limit the presently disclosed systems or methods, their components, acts, or elements. The use herein of “including,” “comprising,” “having,” “containing,” “involving,” and variations thereof is meant to encompass the items listed thereafter and equivalents thereof as well as additional items.

References to “or” may be construed as inclusive so that any terms described using “or” may indicate any of a single, more than one, and all of the described terms. In addition, in the event of inconsistent usages of terms between this document and documents incorporated herein by reference, the term usage in the incorporated features is supplementary to that of this document; for irreconcilable differences, the term usage in this document controls.

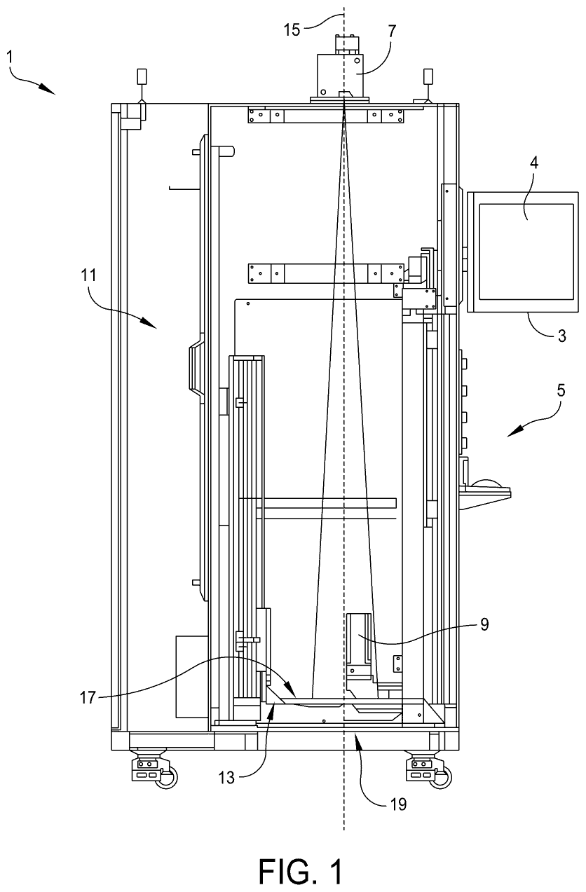

FIG. 1 is a side view of a three-dimensional (3D) model of a traditional X-ray imaging system, which is generally indicated at 1. The X-ray imaging system includes a computer 3 with a display 4 and input devices generally indicated at 5. In some embodiments, the computer 3 is a general-purpose computer and accordingly, detailed discussion of the general-purpose computer will be omitted for the sake of brevity. The display 4 may be a touch screen as known in the art. Examples of input devices 5 include a mouse, a keyboard, and dedicated and customized single buttons. An example of a general-purpose computer is a Thinkpad® from Lenovo.

The X-ray imaging system 1 further includes an X-ray source 7, an optional electric motor 9 and lifting assembly 11, and a support securing structure 13. The support structure 13 is configured to support an object for imaging. The computer 3 controls the operation of the X-ray source 7, along with the height of the support securing structure 13 via the electric motor 9 being mechanically coupled to the lifting assembly 11.

The X-ray source 7 emits X-ray beams along a central axis 15 towards the object(s) to be imaged. The X-ray beams travel in the direction along the central axis 15 as well as at an ever-diverging distance from the central axis 15 as the distance from the X-ray source 7 increases. Some examples of the X-ray imaging system 1 includes a support including a tray 17 made of an X-ray radiolucent material. An X-ray radiolucent material permits X-ray beams to travel through it without significantly interacting with the material to the point where the direction and/or intensity of the beams is affected. As the X-ray beams pass through objects held in place by the tray 17, they are detected by an imaging sensor 19, which sends data from a grid of pixels to the computer 3, which then translates the data into an X-ray image of the objects. The optional lifting assembly 11 allows the tray 17 to be moved between a raised position for ease in loading and unloading objects, and lower positions arranged at a further distance from the X-ray source for imaging.

As mentioned above, X-ray systems can also be used to irradiate products, such as cannabis. Cannabis products may contain mold and/or bacteria contamination, which requires eradication prior to being sold. Irradiation from X-ray exposure has been found to mitigate hazards from such contamination.

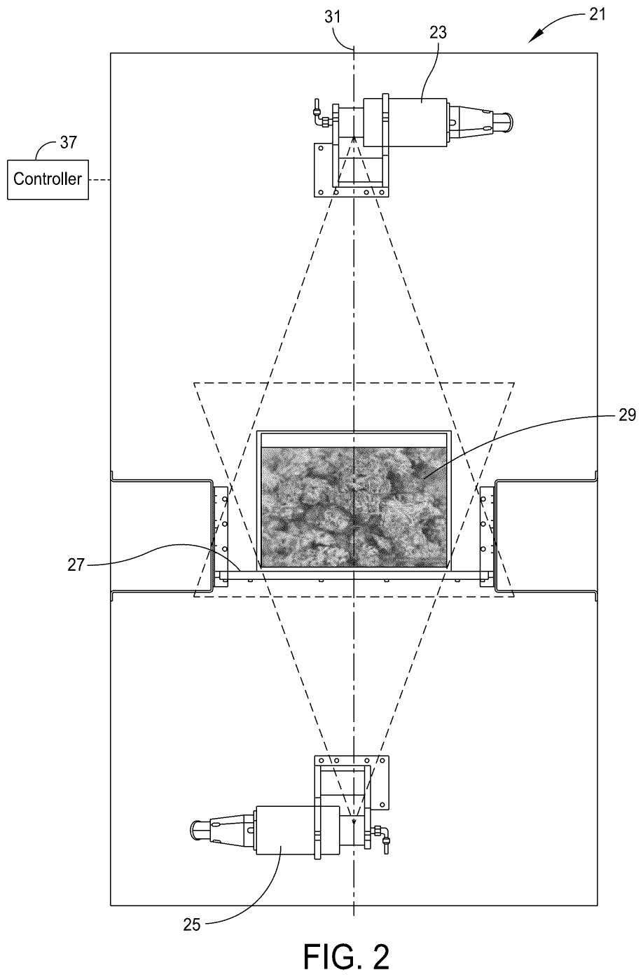

Referring to FIG. 2, an X-ray irradiation system, generally indicated at 21, is configured for two-sided ionizing irradiation with optimized beam orientation to provide greater speed and dose uniformity for the biologic decontamination process. In most aspects, the X-ray irradiation system 21 is configured to operate in a manner that is similar to the operation of X-ray imaging system 1. As shown, the X-ray irradiation system 21 includes a first X-ray source 23, a second X-ray source 25, and a support securing structure 27 configured to support an object 29 requiring decontamination. In one embodiment, the support securing structure 27 can include a processing tray or bin. Further, as discussed herein, the object 29 can embody a cannabis mass. A computer, such as controller 37, controls the operation of the X-ray sources 23, 25, including the intensity, duration, source angle, and distance, to perform irradiation on two sides of the object 29. In one embodiment, the controller 37 can be part of the X-ray irradiation system 21 and can include display and input devices. In another embodiment, the controller 37 can be provided separately from the X-ray irradiation system 21 and coupled to the system to provide external control. The X-ray sources 23, 25 emit X-ray beams along a central axis 31 towards the object 29 to be irradiated. The X-ray beams travel in the direction along the central axis 31 as well as at an ever-diverging distance from the central axis as the distance from the X-ray sources 23, 25 increases.

Ionizing irradiation has been widely applied in a controlled process for mitigating [killing] biologic contaminants in food, pharmaceuticals, medical supplies, and other items such as medicinal and recreational cannabis. A specific dose must be applied to achieve sufficient decontamination, typically defined as number of viable organisms (CFU, or Colony Forming Units).

The entire mass of material being treated must be subjected to the minimum dose to achieve decontamination. However, irradiation at moderate to high levels may have detrimental impact on the product being processed. While it is mandatory to meet minimum dose required, it is desirable to limit the maximum applied dose.

Characteristics of the irradiation delivery apparatus will create high and low relative dose regions within the mass. The ratio of total dose is referred to as dose uniformity ratio (DUR). The specific techniques applied in this claim optimize dose delivery and provide for an exceptionally low DUR.

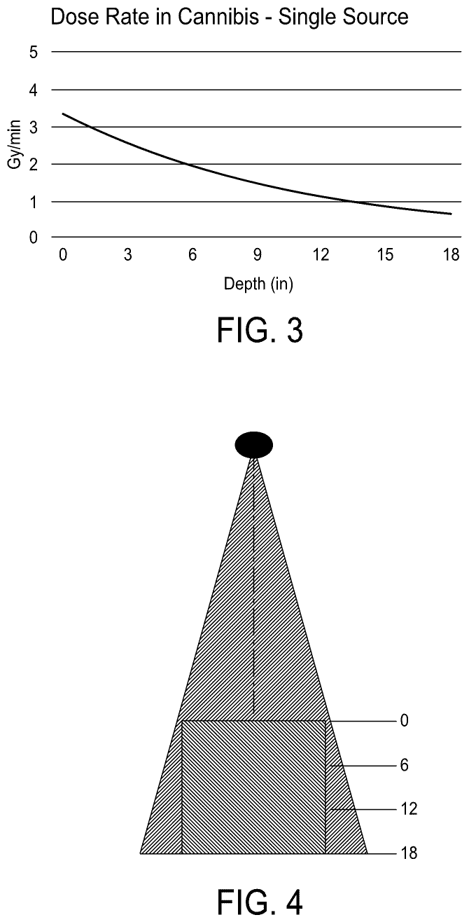

X-ray irradiation has the ability to penetrate deep into, or through the entire mass of the subject material. The X-ray energy is attenuated as it travels through the mass. The dose rate is highest at the point of entry, and exponentially declines as it travels through the mass. However, limiting the product thickness creates an attenuation curve that approximates a linear relationship. FIGS. 3 and 4 both illustrate how dose rate in cannabis decline through the depth of the cannabis mass. Specifically, FIG. 3 illustrates a dose rate in cannabis in which the dose rate at entry is approximately 3.4 Gy/min at the point of entry of the cannabis mass and the dose rate is approximately 0.7 Gy/min at a depth of 18-inches. FIG. 4 illustrates the application of the X-ray energy through the mass. As shown, the dose rate drops precipitously through the thickness of object being irradiated. The present disclosure describes providing equal and opposite irradiation from two sides results in a very low DUR through the cannabis mass.

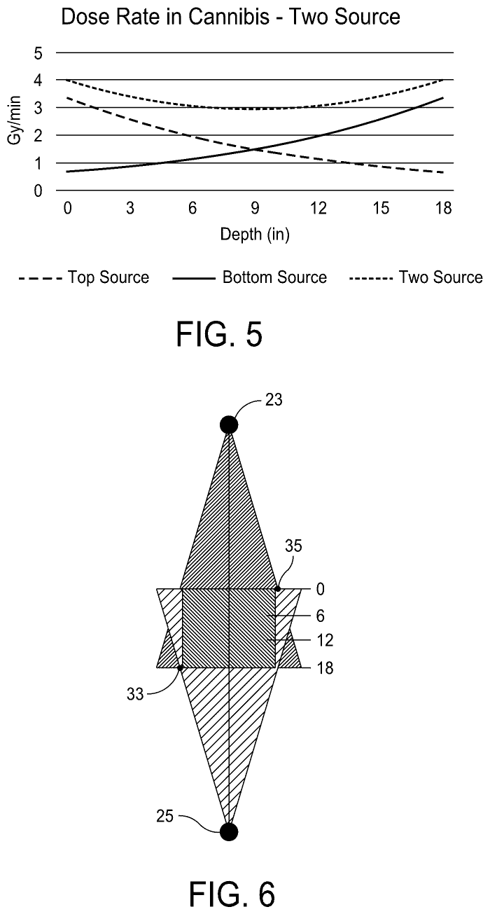

Referring to FIGS. 5 and 6, FIG. 5 illustrates dose rates with two sources, e.g., X-ray source 23 and X-ray source 25, in cannabis and FIG. 6 illustrates X-ray energy through two sides of an object, i.e., a cannabis mass. FIG. 5 illustrates a dose rate from a first (top) X-ray source, e.g., X-ray source 23 for system 21, in cannabis in which the dose rate is approximately 3.4 Gy/min at the point of entry of the cannabis (top) mass and the dose rate is approximately 0.65 Gy/min at a depth of 18-inches (bottom of mass). FIG. 5 further illustrates a dose rate from a second (bottom) X-ray source, e.g., X-ray source 25 for system 21, in cannabis in which the dose rate is approximately 3.4 Gy/min the bottom of the cannabis mass and the dose rate is approximately 0.65 Gy/min at the top of the mass. The combined dose rate of the top source of irradiation and the bottom source of irradiation results in a dose rate between approximately 2.95 Gy/min to approximately 4.0 Gy/min through the cannabis mass. The combined dose rate produces a relatively homogeneous result, rather than one side of the mass having a much greater dose than the other side of the mass.

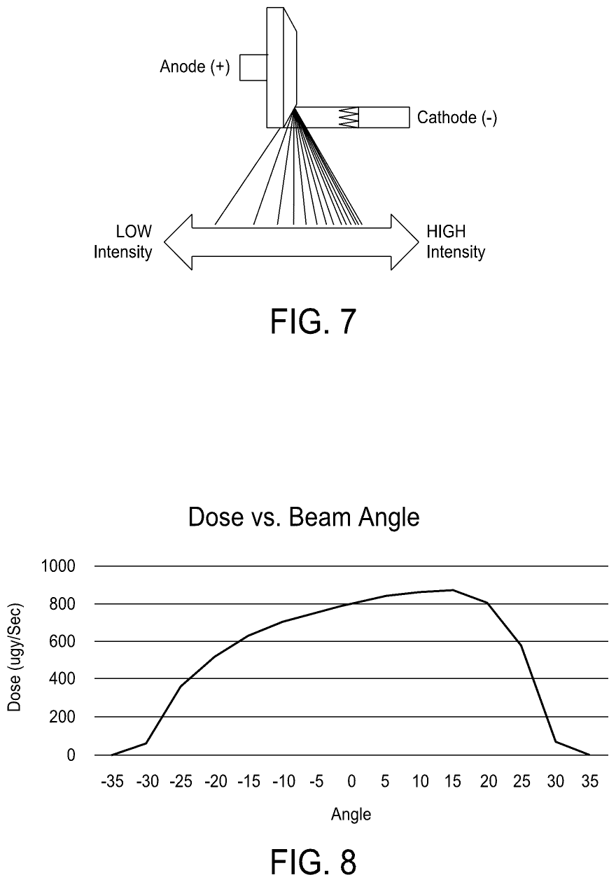

Referring to FIGS. 7 and 8, to optimize beam orientation, a typical X-ray sources generate a cone shaped emission of X-ray energy, sometimes referred to as a cone beam X-ray. The total energy (flux) emitted varies based on the exit angle from the X-ray window. Flux is generally higher in the center of the cone (0 degree) and decreases at increased angles.

Most high-powered X-ray sources use a reflective target. Energy emitted from reflective target sources will typically decline faster on one side of the cone due to a phenomenon known as the heel effect. Unless accounted for in the design, lower energy at the heel will adversely affect DUR.

FIG. 7 illustrates a cathode generating an energy source directed to an anode, which reflects the energy source to produce X-ray irradiation. As shown, at 0-degree angle, high intensity irradiation is produced. Even a relatively small angle, e.g., 25-degree angle, drastically reduces the intensity of the irradiation source. This reduction of intensity of irradiation at greater angles is shown in FIG. 8.

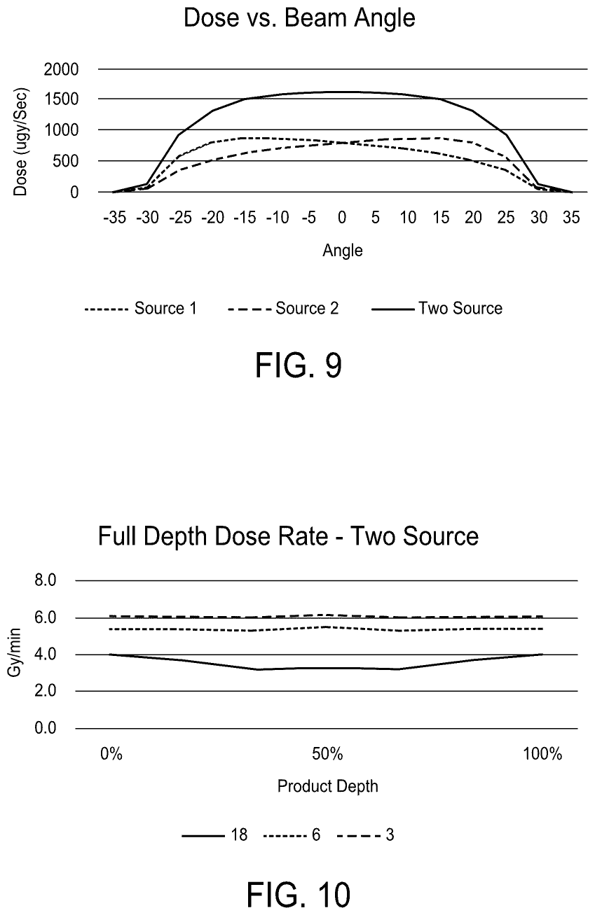

Referring to FIG. 9, the application of two source irradiation as disclosed herein, e.g., X-ray source 23 and X-ray source 25, with the heel of the sources of irradiation oriented in opposite directions results in a beneficial additive affect. As shown, combining two-sided irradiation to account for attenuation with opposed heel orientation provides substantial improvement to the DUR. The combined effect produces a significant increase in total dose, from approximately 850 uGy/sec to approximately 1600 uGy/sec at a distance of 1.0 m from the source. The combined dose rate of the first (top) source of irradiation and the second (bottom) source of irradiation results in a dose rate that is more uniform and provides nearly double the dose rate of either the first source of irradiation or the second source of irradiation.

Embodiments of the methods disclosed herein can be configured to optimize an X-ray decontamination process speed with reduce risk of over exposure through use of in situ dose monitoring.

Referring to FIG. 10, in one embodiment, a dose rate for X-ray decontamination is dependent on the density of the product and the total thickness of product to be decontaminated. To guaranty proper dose (therefore successful decontamination without over processing) these factors must be accounted for with each batch of product as it is loaded into the X-ray irradiation system 21. As shown, the dose rate is generally consistent throughout the depth of the product to be decontaminated. This is attributable to providing sources of irradiation on both sides of the product, e.g., the first X-ray source 23 and the second X-ray source 25.

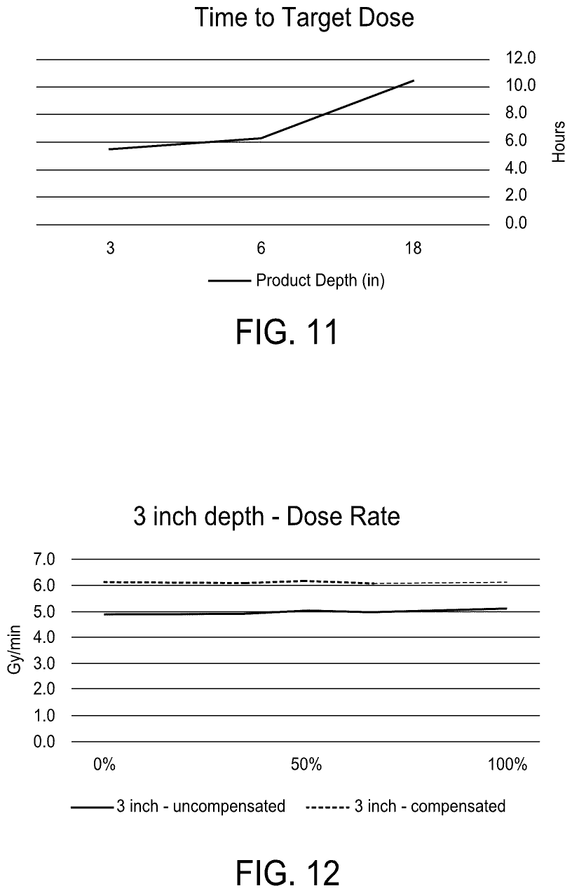

Referring to FIG. 11, an in situ dose monitor or probe is designed to independently measure unattenuated dose rate and fully attenuated dose rate. The actual dose rates are then used to calculate the ideal time to achieve required total dose. As shown, for a product having a thickness of 18-inches, the time to target dose is increased from 5.5 hours for a three-inch thick product to 10.4 hours for an 18-inch-thick product. For example, the dose rate is highly dependent on a depth of the product on the support 27, such as a minimum dose rate for an 18-inch depth can be 3.2 Gy/min, 5.3 Gy/min or 6.1 Gy/min.

The dose probe is monitored throughout the process to integrate the actual delivered total dose, assuring accuracy regardless of product depth, product density, moisture content, and variations in containers and/or product packaging. Referring back to FIG. 6, a first probe 33 can be positioned to represent full attenuation of the first X-ray source 23 and a second probe 35 can be positioned to represent full attenuation of the second X-ray source 25. The probes 33, 35 are provided in the paths of the X-ray sources 23, 25 and configured to measure unattenuated and attenuated energy. Based on information obtained, the X-ray irradiation system 21 can be configured to automatically determine dose rate and compensate for product depth and compensate for variation in dose attenuation, including density, moisture content, and impact of container and/or packaging. The probes 33, 35 enable the X-ray irradiation system 21 to provide real-time feedback to assure a proper dose is applied to the object 29.

Embodiments of the methods disclosed herein provide a method to automatically adjust the positioning of the X-Ray sources 23, 25 of the X-ray irradiation system 21 with respect to the product.

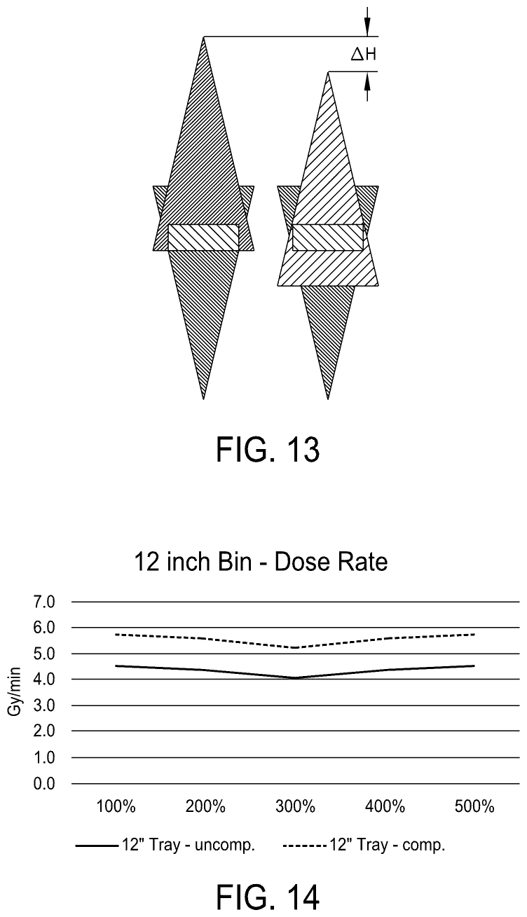

Referring to FIGS. 12 and 13, dose rate and DUR are highly dependent on the distance between the X-ray source and the product. The dose rate is inversely proportional to the square of the distance. The source should be positioned as close to the product as possible such that the product fits completely within the high intensity cone of X-ray flux.

The X-ray system must be designed to accommodate the maximum volume of product to be decontaminated. When the full volume is not used, a fixed X-ray source orientation will result in sub-optimized process. The dose rate, while faster than it would be while running full volume, will not be optimized. Additionally, the DUR will be adversely affected.

Automatically adjusting the source position to match the product depth will improve the dose rate and provide improved DUR through the product depth. FIG. 12 shows a lower dose rate for a product that is not compensated (4.9 Gy/min) versus a higher dose rate for a product that is compensated (6.1 Gy/min). FIG. 13 shows positioning the top source of irradiation closer to the product, e.g., 3-inches, to improve the efficiency of the process.

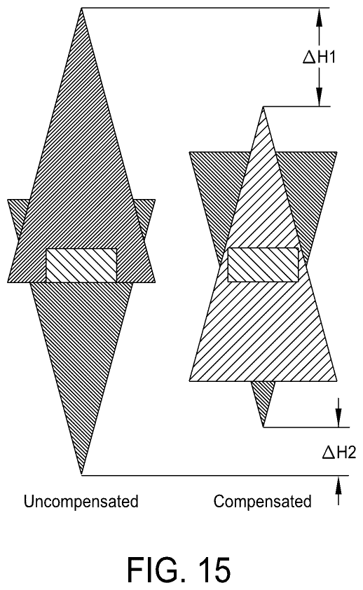

Referring to FIGS. 14 and 15, changes in length and width will also impact efficiency of the process. To optimize processing speed, the sources, e.g., the first X-ray source 23 and the second X-ray source for the X-ray irradiation system, should be as close as possible to properly irradiate to the entire volume, but not to waste energy by irradiating empty space. Both sources should be automatically adjusted for the product bin dimensions. FIG. 14 shows a lower dose rate for a product that is not compensated (approximately 4.1 Gy/min) versus a higher dose rate for a product that is compensated (approximately 5.2 Gy/min). FIG. 15 shows positioning the top source of irradiation and the bottom source of irradiation closer to the product, e.g., 12-inches, to improve the efficiency of the process.

In some embodiments, the position of the sources 23, 25 can be independently controlled. For example, the first (top) X-ray source 23 can be positioned relative to the top of the object 29 product located on the support 27. Similarly, the second (bottom) X-ray 25 source can be positioned relative to the bottom of the object 29 located on the support 27.

It should be understood that the system and the methods disclosed herein can be utilized to irradiate any type of biological or organic product. For example, and not meant to be limiting, the system and methods described can be applied to foods, spices, pharmaceuticals, medical devices, and packaging, as well as cannabis. Further, the system and methods described can be used to deactivate microorganisms, such as bacteria, fungi, viruses and spores. Further, the dose rates and the uniformity data provided herein are examples for a specific configuration and product to be decontaminated. Dose rates and uniformity may vary based on X-ray sources used, X-ray parameters, volume of product, and density of product. It is expected that resulting improvements are similar to those presented herein.

Having thus described several aspects of at least one embodiment, it is to be appreciated various alterations, modifications, and improvements will readily occur to those skilled in the art. Such alterations, modifications, and improvements are intended to be part of, and within the spirit and scope of, this disclosure. Accordingly, the foregoing description and drawings are by way of example only.

What is claimed is:

1. An X-ray irradiation system comprising:
a support for an object requiring decontamination;
a first source of irradiation directed to a top side of the object;
a second source of irradiation directed to a bottom side of the object; and
a controller configured to control a dose rate and a time of each of the first source and the second source to optimize decontamination of the object.

2. The X-ray irradiation system of claim 1, wherein the first source of irradiation is configured to produce a dose rate of approximately 3.4 Gy/min at the point of entry (top) of the object and a dose rate of approximately 0.7 Gy/min at a depth of 18-inches (bottom) of the object and the second source of irradiation is configured to produce a dose rate of approximately 3.4 Gy/min at the point of entry (bottom) of the object and a dose rate is approximately 2.95 Gy/min at a depth of 0-inches (top) of the object.

3. The X-ray irradiation system of claim 2, wherein a combined dose rate of the first source of irradiation and the second source of irradiation results in a dose rate between approximately 2.95 Gy/min to approximately 4.0 Gy/min through the object.

4. The X-ray irradiation system of claim 1, wherein the object is a cannabis mass.

5. The X-ray irradiation system of claim 4, wherein the first source of irradiation is configured to direct irradiation to a top surface of the cannabis mass and the second source of irradiation is configured to direct irradiation to a bottom surface of the cannabis mass.

6. The X-ray irradiation system of claim 1, wherein the first source of irradiation and the second source of irradiation are each configured to be adjusted to adjust an angle of the source of irradiation with respect to the object.

7. The X-ray irradiation system of claim 6, wherein the first source of irradiation and the second source of irradiation are oriented in opposite directions.

8. The X-ray irradiation system of claim 7, wherein a combined dose rate of the first source of irradiation and the second source of irradiation results in a dose rate that is more uniform and provides nearly double the dose rate of the first source of irradiation or the second source of irradiation.

9. The X-ray irradiation system of claim 1, further comprising a dose probe configured to monitor the object to obtain a delivered total dose applied to the object.

10. The X-ray irradiation system of claim 1, wherein at least one of the first source of irradiation and the second source of irradiation is configured to be adjusted to adjust a distance of the source of irradiation with respect to the object.

11. The X-ray irradiation system of claim 10, wherein both the first source of irradiation and the second source of irradiation are configured to be adjusted to adjust the distance of the source of irradiation with respect to the object.

12. The X-ray irradiation system of claim 10, wherein a process speed is optimized when adjusting the distance of at least one of the first source of irradiation and the second source of irradiation.

13. A method of irradiating an object with an X-ray irradiation system, the method comprising:
applying a first source of irradiation directed to a top side of the object;
applying a second source of irradiation directed to a bottom side of the object; and
controlling a dose rate and a time of each of the first source and the second source to optimize decontamination of the object.

14. The method of claim 13, wherein applying the first source of irradiation includes a dose rate of approximately 3.4 Gy/min at the point of entry (top) of the object and a dose rate of approximately 0.7 Gy/min at a depth of 18-inches (bottom) of the object and applying the second source of irradiation includes a dose rate of approximately 3.4 Gy/min at the point of entry (bottom) of the object and a dose rate is approximately 0.7 Gy/min at a depth of 0-inches (top) of the object.

15. The method of claim 14, wherein a combined dose rate of the first source of irradiation and the second source of irradiation results in a dose rate between approximately 2.95 Gy/min to approximately 4.0 Gy/min through the object.

16. The method of claim 13, wherein the object is a cannabis mass.

17. The method of claim 16, wherein applying the first source of irradiation includes directing irradiation to a top surface of the cannabis mass and applying the second source of irradiation includes directing irradiation to a bottom surface of the cannabis mass.

18. The method of claim 13, further comprising adjusting an angle of one of the first source of irradiation and the second source of irradiation with respect to the object.

19. The method of claim 18, wherein the first source of irradiation and the second source of irradiation are oriented in opposite directions.

20. The method of claim 19, wherein a combined dose rate of the first source of irradiation and the second source of irradiation results in a dose rate that is more uniform and provides nearly double the dose rate of the first source of irradiation or the second source of irradiation.

21. The method of claim 13, further comprising positioning a dose in the object to monitor the object to obtain a delivered total dose applied to the object.

22. The method of claim 1, further comprising adjusting a distance of at least one of the first source of irradiation and the second source of irradiation with respect to the object.

23. The method of claim 22, further comprising optimizing a process speed when adjusting the distance of at least one of the first source of irradiation and the second source of irradiation.
